(19) United States
(12) Patent Application Publication
(10) Pub. No.: US 2025/0095792 A1
(43) Pub. Date: Mar. 20, 2025

(54) METHOD FOR IDENTIFYING TRANSFORMATION PRODUCTS OF ANTIBIOTICS FROM KNOWN AND POTENTIAL UNKNOWN TRANSFORMATION PATHWAYS

(71) Applicants: Peking University, Beijing (CN); Nanyang Normal University, Nanyang City (CN)

(72) Inventors: Weiling SUN, Beijing (CN); Jingrun HU, Beijing (CN); Yi LIU, Beijing (CN); Minjie DENG, Beijing (CN); Mingfei JI, Beijing (CN); Yuying LI, Beijing (CN)

(21) Appl. No.: 18/735,796

(22) Filed: Jun. 6, 2024

(30) Foreign Application Priority Data
Sep. 5, 2023 (CN) 202311132075.0

(51) Int. Cl. G16C 20/20; G01N 30/02; G01N 30/72; G01N 30/86

(52) U.S. Cl. CPC G16C 20/20; G01N 30/7233; G01N 30/8631; G01N 2030/027

(57) ABSTRACT

A method for identifying transformation products of antibiotics from known and potential unknown transformation pathways includes: step 1: collecting samples, extracting antibiotics and transformation products thereof, and obtaining non-target data by ultra-high performance liquid chromatography-high resolution mass spectrometry; step 2: identifying transformation products of antibiotics from known and unknown transformation pathways; step 3: generating a list of candidate transformation products and annotating their structures; step 4: extracting feature fragments based on structure annotations of the transformation products obtained in step 3, and searching for spectra with the feature fragment, and supplementing the transformation products from unknown transformation pathway; and step 5: obtaining a final list of identified products based on annotated structures.

### CROSS-REFERENCE TO RELATED APPLICATION

This patent application claims the benefit and priority of Chinese Patent Application No. 202311132075.0, filed with the China National Intellectual Property Administration on Sep. 5, 2023, the disclosure of which is incorporated by reference herein in its entirety as part of the present application.

### TECHNICAL FIELD

The present disclosure belongs to the technical field of detection and analysis of antibiotics, and more particularly, to a method for identifying transformation products of antibiotics from known and potential unknown transformation pathways.

### BACKGROUND

Antibiotics have been widely applied to the prevention and treatment of human and animal diseases. Antibiotics may be discharged into surface water in multiple ways such as sewage, industrial wastewater, and surface runoff. Antibiotics may be metabolized or transformed into various products in the bodies of a human and an animal, during water treatment processes, or in natural water. Studies have found that transformation products of certain antibiotics in effluent of sewage treatment plants or in natural environment are higher in content and greater in persistence, mobility, and toxicity than their parent compounds thereof. For example, two metabolites (sulfamethoxazole hydroxylamine and 4-nitrososulfamethoxazole) of sulfamethoxazole have higher antimicrobial activity than their parent compound. Therefore, it is quite necessary to comprehensively identify transformation products of antibiotics in the natural environment.

An existing study has discussed transformation pathways of some common antibiotics and identified part of transformation products thereof. For example, laboratory studies have identified 52 transformation products of amoxicillin through photolysis and hydrolysis, including typical transformation products amoxicilloic acid and amoxicillin piperazine-2,5-dione. The transformation pathways obtained by these laboratory experiments and the identified transformation products lay a certain foundation for the identification of transformation products of antibiotics in a complex environmental sample.

It needs to be noted that there are hundreds of parent antibiotics currently detected in water environment, and limited types of parent antibiotics are involved in laboratory studies on transformation products of antibiotics, which may not completely cover these detected parent antibiotics. Moreover, since the actual environmental system is more complex than a simulated system in a laboratory, screening transformation products of antibiotics in actual samples by only relying on known pathways and products determined in the laboratory will inevitably result in potential omission of transformation products. Therefore, to comprehensively identify transformation products of antibiotics, it is quite necessary to integrate both known and potential unknown transformation pathways.

Prior art document 1 (CN107884507B) discloses a method for simultaneously and quickly screening pesticides, drugs, and transformation products thereof in wastewater, where identifying transformation products includes the following main steps: 1) predicting transformation products of parent compounds of pesticides and drugs that have been identified, to obtain possible transformation products of the parent compounds, and performing suspected screening; and 2) performing non-target screening with a most frequently occurring fragment ion in the parent compounds and the transformation products as a diagnostic ion. The prior art document 1 has the following disadvantages: the basis of the non-target screening is known transformation products or unknown transformation products under known transformation pathways predicted by software, and the information on transformation products under unknown transformation pathways may be neglected.

Prior art document 2 (CN111707741A) discloses a non-target identification method for transformation products of trace organic pollutants in environmental media, where a molecular network method is mainly used to identify trace organic pollutants in various environmental media, including identifying transformation products of drugs and personal care products, persistent organic pollutants, polychlorinated biphenyls, polycyclic aromatic hydrocarbons, organic pesticides, and perfluorinated compound organic pollutants. The non-target screening based on molecular network can specifically identify mass spectra with similar structures and identify new unknown products without related information on transformation products. The prior art document 2 has the following disadvantage: the lack of related information about the transformation process may result in missing of products in the identification process.

### SUMMARY

To solve the shortages present in the prior art, the present disclosure provides a method for identifying transformation products of antibiotics from known and potential unknown transformation pathways to comprehensively identify transformation products of antibiotics in a natural sample.

The present disclosure adopts the following technical solutions. The present disclosure provides a method for identifying transformation products of antibiotics from known and potential unknown transformation pathways, including:
step 1: collecting samples from different sampling sites in a target area, extracting antibiotics and transformation products thereof, and collecting non-target data by ultra-high performance liquid chromatography-high resolution mass spectrometry, and preprocessing the data to obtain a peak list;
step 2: based on the mass spectrometry data obtained in step 1, performing step A of identifying transformation products of the antibiotics from known transformation pathways and performing step B of identifying transformation products of the antibiotics from unknown transformation pathways;
where step A includes: collecting structural information of known antibiotics, establishing an information list of parent antibiotics, and establishing a suspect list of transformation products; and step B includes: establishing a molecular network, extracting all features of a sub-network where a parent is located, and obtaining the transformation products from the unknown transformation pathways;
step 3: obtaining a list of candidate transformation products based on the transformation products from the known and unknown transformation pathways obtained in step 2, and annotating structures of the transformation products;
step 4: extracting feature fragments based on structure annotations of the transformation products obtained in step 3, and searching the peak list with the feature fragments, and supplementing the transformation product from the unknown transformation pathways; and
step 5: obtaining a final list of identified products after annotating the structures of transformation products.

Preferably, step A includes:
step A.1: collecting the structural information of the known antibiotics and establishing the information list of parent antibiotics;
step A.2: for a parent antibiotic obtained in step A.1, obtaining the suspect list of all transformation products; and
step A.3: merging the predicted transformation products obtained in step A.2.

Preferably, in step A.2, the suspect list of the transformation products established according to the known transformation pathways includes known transformation products and predicted transformation products from the known transformation pathways.

Preferably, step A.3 includes: removing a transformation product having a similarity of less than a set value and having the same molecular descriptor with a parent structure for merging the prediction results of the transformation products obtained in step A.2.

Preferably, step B specifically includes:
step B.1: preprocessing the mass spectrometry data obtained in step 1 to obtain a feature quantification table and a mass spectrometry (MS)/MS spectral summary;
step B.2: establishing the molecular network with the feature quantification table and an MS/MS spectral summary obtained in step B.1; and
step B.3: extracting mass spectrum peak information of transformation products of the parent antibiotic from the unknown transformation pathways using the molecular network obtained in step B.2.

Preferably, step B.2 includes: exporting the feature quantification table and the MS/MS spectral summary obtained in step B.1 to Global Natural Products Social Molecular Networking platform, creating the molecular network using a feature-based molecular networking workflow, and setting a minimum matched fragment ion and a cosine score.

Preferably, step B.3 includes: in a graph of the molecular network established in step B.2, locating an area where the parent antibiotic and the known transformation products are present based on a mass-to-charge ratio of a precursor ion shown on a node in the molecular network, thereby extracting all the features of the sub-network, and obtaining mass spectra of the transformation products of the parent antibiotic from the unknown transformation pathways.

Preferably, step 3 includes: annotating the structure of the transformation product from the unknown transformation pathways and assigning corresponding confidence levels 1 to 5 to the structures of all the transformation products.

Preferably, step 4 includes: using fragment ions frequently occurring in the parent antibiotic and transformation products screened from the samples as the feature fragments.

Preferably, step 5 includes: selecting the transformation products at the confidence levels 1 to 3 to obtain the final list of identified products.

Compared with the prior art, the present disclosure has at least the following beneficial effects:
(1) The present disclosure allows for the identification of transformation products of antibiotics from both known and potential unknown transformation pathways by combining the suspect list of the transformation products and the molecular network.
(2) Feature fragments are extracted from the results of all the transformation products on the basis of the suspect list and the screening result of the molecular network. The feature fragments include the information of the known and potential unknown transformation pathways. After conducting suspect screening and molecular network analysis, non-target screening is further performed using the obtained feature fragments, which enhances the scope of detection and facilitates a more thorough screening of antibiotic transformation products.

### BRIEF DESCRIPTION OF THE DRAWINGS

FIG. 1 is a flowchart of a method for identifying transformation products of antibiotics from known and potential unknown transformation pathways provided in the present disclosure.

### DETAILED DESCRIPTION OF THE EMBODIMENTS

In order to make the objective, technical solutions, and advantages of the present disclosure clearer, the technical solutions of the present disclosure will be clearly and completely described below in conjunction with the accompanying drawings in embodiments of the present disclosure. The embodiments described herein are merely some rather than all of the embodiments of the present disclosure. All other embodiments derived based on the spirit of the present disclosure by a person of ordinary skill in the art without creative efforts shall fall within the protection scope of the present disclosure.

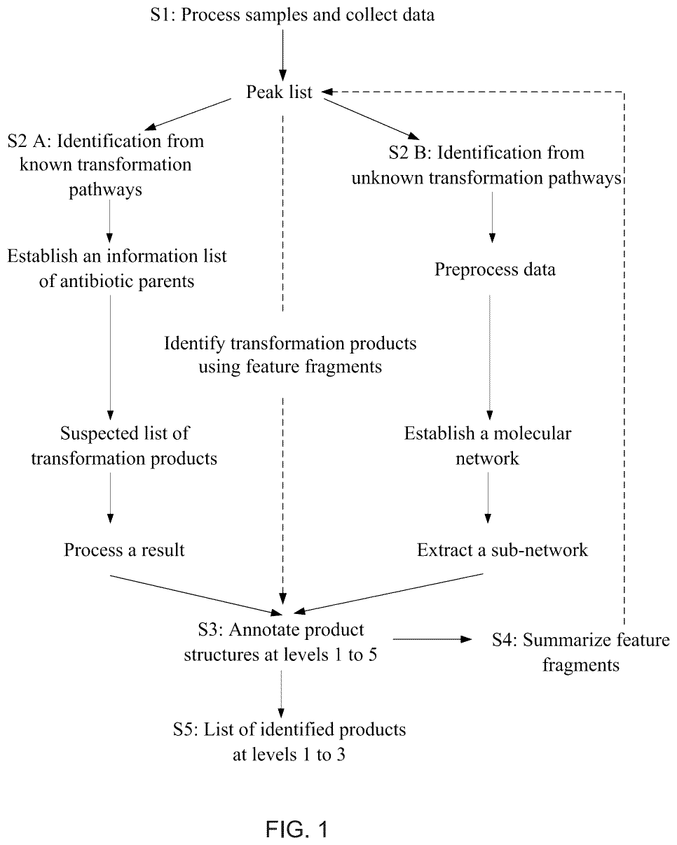

As shown in FIG. 1, Example 1 of the present disclosure provides a method for identifying transformation products of antibiotics from known and potential unknown transformation pathways, including the following steps.

Step 1: water samples are collected from different sampling sites in a target area, antibiotics and their transformation products thereof are extracted, and non-target data is collected by ultra-high performance liquid chromatography-high resolution mass spectrometry and preprocessed to obtain a peak list.

In a preferred but non-limiting embodiment of the present disclosure, step 1 specifically includes the following steps.

Step 1.1: the samples are collected from different sampling sites in the target area and preprocessed to achieve the purposes of enrichment and purification, and the antibiotics and their transformation products thereof are extracted.

Further preferably, 9 surface water samples are collected, which are numbered as S1 to S9. The samples are collected in clean 1 L brown glass bottles, immediately delivered to a laboratory by using an ice cooler, and stored in a dark environment at 4° C. Within 24 hours after collection, particles in the samples are removed by filtration using a filter membrane. Antibiotics and their transformation products in the samples are extracted by solid phase extraction, and then subjected to elution, nitrogen purging, and constant volume preparation. The number of the samples may be arbitrary, and the operations on the samples are the same.

Step 1.2: the non-target data is collected by ultra-high performance liquid chromatography-high resolution mass spectrometry from the samples obtained in step 1.1 and preprocessed to obtain the peak list.

Further preferably, non-target analysis is performed using an ultra-high performance liquid chromatography-high-resolution mass spectrometer.

More preferably, mass spectrometry (MS) full scanning is performed in both positive and negative ionization modes, with a mass range of m/z 100-1500 and a resolution of 140,000, and data-dependent MS/MS collection is performed for 3 times, with a resolution of 17,500.

Step 2: based on the mass spectrometry data obtained in step 1, step A of identifying a transformation product of the antibiotic from known transformation pathways and step B of identifying a transformation product of the antibiotic from unknown transformation pathways are performed.

Step A includes: collect structural information of known antibiotics, establish an information list of parent antibiotics, and establish a suspect list of transformation products; and step B includes: establish a molecular network, extract all features of a sub-network where a parent antibiotic is located, and obtain the transformation products from the unknown transformation pathway.

It needs to be noted that an order of performing step A and step B is not fixed, and step A or step B may be performed firstly, or step A and step B may be performed simultaneously.

In a preferred but non-limiting embodiment of the present disclosure, step A specifically includes the following steps.

Step A.1: the structural information of the known antibiotics is collected and the information list of parent antibiotics is established.

Specifically, the information list of the parent antibiotics is compared with the mass spectra to find out the parent antibiotics in the sample. The information list of known parent antibiotics includes, but is not limited to, existing lists of antibiotics from various literature, parent antibiotics in NORMAN Suspect List Exchange database, China Drug Statistical Yearbook, and structural information of all commercially available antibiotics.

It needs to be noted that the range of the information list of parent antibiotics may be selected according to an actual sample. For example, for an aquaculture wastewater sample, a list of veterinary antibiotics is established and then subsequent operation steps may be performed.

step A.2: for a parent antibiotic obtained in step A.1, the suspect list of all transformation products is obtained.

It needs to be noted that the transformation products of a parent antibiotic may be predicted using transformation pathways that have been determined in previous studies or using any prediction tool according to known transformation pathways. Prior to the filing date of the present disclosure, preferably but not limited to, the transformation products of the parent antibiotics present in the sample from the known transformation pathway are predicted using BioTransformer 3.0. After the filing date of the present disclosure, performing step A.2 using any new prediction tool and predicting transformation products of a parent antibiotic from a known transformation pathway may also fall within the scope of the present disclosure. BioTransformer3.0 is merely an example prediction tool rather than a unique tool.

step A.3: prediction results of the transformation products obtained in step A.2 are merged.

Further preferably, a transformation product having a similarity of less than a set value and having the same molecular descriptor with its parent structure is removed for merging the prediction results of the transformation products obtained in step A.2.

More preferably, the transformation products having a similarity of less than 50% and having the same molecular descriptor with its parent structure are removed in a batch processing mode using R language patRoon 2.0 package.

In a preferred but non-limiting embodiment of the present disclosure, step B specifically includes the following steps.

Step B.1: the mass spectrometry data obtained in step 1 is preprocessed to obtain a feature quantification table and an MS/MS spectral summary.

Further preferably, the original mass spectrometry data is converted to an ABF file format by a Reifycs basic file converter and preprocessed in MS-DIAL 4.9054 software, and finally exported as the feature quantification table (TXT format) and an MS/MS spectral summary (MGF format).

Step B.2: the molecular network is established with the feature quantification table and the MS/MS spectral summary obtained in step B.1.

Further preferably, the feature quantification table and the MS/MS spectral summary obtained in step B.1 are output to Global Natural Products Social Molecular Networking (GNPS) platform (https://gnps.ucsd.edu), and the molecular network is established using a feature-based molecular networking workflow.

More preferably, a minimum matched fragment ion and a cosine score are set to 5 and 0.6, respectively. Finally, the molecular network is visualized using Cytoscape3.9.1. The cosine score refers to a cosine similarity of a pair of MS/MS spectra.

Step B.3: mass spectrum peak information of transformation products of the parent antibiotic from the unknown transformation pathway is extracted using the molecular network obtained in step B.2.

Further preferably, in a graph of the molecular network established in step B.2, an area where the parent antibiotic and the known transformation products are present is located based on a mass-to-charge ratio of a precursor ion shown on a node in the molecular network, thereby extracting all the features (having a great similarity of second order spectra) of the sub-network, which may be the mass spectra of the transformation products of the parent antibiotic from the unknown transformation pathways.

Step 3: a list of candidate transformation products is obtained based on the transformation products from the known and unknown transformation pathways obtained in step 2, and structures of the transformation products are annotated.

In a preferred but non-limiting embodiment of the present disclosure, the structure of the transformation product from the unknown pathway is analyzed by a computer prediction mass spectrum fragmentation tool, for example, but not limited to, MetFrag and Mass Frontier 7.0. Corresponding confidence levels 1 to 5 are assigned to the structures of all the transformation products.

Step 4: a feature fragment is extracted based on structure annotations of the transformation products obtained in step 3, and a search is made in the peak list with the feature fragments, and the transformation products from the unknown transformation pathway are supplemented.

In a preferred but non-limiting embodiment of the present disclosure, a fragment ion most frequently occurring in the parent antibiotic and transformation products is used as the feature fragment.

Further preferably, the search is made with a set mass deviation and an ion abundance. The features including the feature fragment in the MS/MS spectrum are preferably used as the candidate transformation products from the unknown transformation pathways for further structural analysis.

More preferably, when the feature fragment is searched, the mass deviation is 0.01 Da and the ion abundance is 10%.

Step 5: a final list of identified products is obtained after the structures are annotated based on transformation product results.

In a preferred but non-limiting embodiment of the present disclosure, the transformation products at levels 1 to 3 are selected to obtain the final list of identified products.

In order to introduce the technical solutions of the present disclosure and the beneficial technical effects that can be achieved more clearly, an example of the present disclosure is described below, which includes the following steps.

Surface water samples are collected at 9 points in a target area, numbered as S1 to S9, collected in clean 1 L brown glass bottles, immediately put into cold storage and delivered to a laboratory, and stored in a dark environment at 4° C. Within 24 hours after collection, particles in the samples are removed by filtration using a filter membrane. Antibiotics and their transformation products are extracted by solid phase extraction, and then subjected to elution, nitrogen purging and evaporation, and constant volume preparation. Antibiotics and their transformation products thereof are extracted.

Non-target analysis is performed using an ultra-high performance liquid chromatography high-resolution mass spectrometer. MS full scanning is performed in both positive and negative ionization modes, with a mass range of m/z 100-1500 and a resolution of 140,000, and data-dependent MS/MS collection is performed for 3 times, with a resolution of 17,500.

A list of parent antibiotics including 663 compounds is established. Transformation products of the obtained 663 parent antibiotics from the known transformation pathways are predicted using BioTransformer 3.0, obtaining 80924 predicted transformation products.

The transformation products having the similarity of less than 50% and having the same molecular descriptor with the parent structure are removed in a batch processing mode using R language patRoon 2.0 package, and a total of 882 suspected transformation products are used for subsequent screening.

The original mass spectrometry data is converted to an ABF file format by a Reifycs basic file converter and preprocessed in MS-DIAL 4.9054 software, and finally exported as a feature quantification table in the TXT format and an MS/MS spectral summary in the MGF format.

The feature quantification table in the TXT format and the MS/MS spectral summary in the MGF format are output to the GNPS platform. A molecular network is established using the FBMN workflow. The minimum matched fragment ion and the cosine score are set to 5 and 0.6, respectively. Finally, the molecular network is visualized using Cytoscape 3.9.1.

In the graph of the molecular network established, an area where the parent antibiotic and the known transformation products are present is located based on a mass-to-charge ratio of a precursor ion shown on a node in the molecular network, thereby extracting all the features of the sub-network, which may be the mass spectra of the transformation products of the parent antibiotic from the unknown transformation pathways. The information of a total of 138 mass spectra is collected.

A list of candidate transformation products is obtained based on the transformation products from the known and unknown transformation pathways, and the structures of the transformation products are annotated. The structure of the transformation products from the unknown pathways is analyzed by MetFrag and Mass Frontier 7.0. Corresponding confidence levels 1 to 5 are assigned to the structures of all the transformation products.

A fragment ion most frequently occurring in the parent antibiotic and transformation products is used as the feature fragment. The feature fragment is searched using “MS/MS fragment searcher” in MS-DIAL4.9054. A mass deviation is 0.01 Da and an ion abundance is 10%. The features including the feature fragment in the MS/MS spectrum are preferably used as the candidate transformation products from the unknown transformation pathways for further structural analysis.

After the structures are annotated based on transformation products of the above steps, the transformation products at levels 1 to 3 are selected, and a total of 176 transformation products of antibiotics at confidence levels 1 to 3 are screened from the samples.

The present disclosure may be a system, method, and/or computer program product. The computer program product may include a computer-readable storage medium storing computer-readable program instructions for enabling a processor to implement various aspects of the present disclosure.

It should be noted that the above embodiments are merely intended to describe the technical solutions of the present disclosure, rather than to limit the present disclosure. Although the present disclosure is described in detail with reference to the above embodiments, a person of ordinary skill in the art may still make modifications or equivalent substitutions to the specific implementations of the present disclosure without departing from the spirit and scope of the present disclosure. However, these modifications or equivalent substitutions should fall within the protection scope of the claims of the present disclosure.

What is claimed is:

1. A method for identifying transformation products of antibiotics from known and potential unknown transformation pathways, comprising:
step 1: collecting samples from different sampling sites in a target area, extracting antibiotics and transformation products thereof, and collecting non-target data by ultra-high performance liquid chromatography-high resolution mass spectrometry to obtain a peak list;
step 2: based on the mass spectrometry data obtained in step 1, performing step A of identifying transformation products of the antibiotics from known transformation pathways and performing step B of identifying transformation products of the antibiotics from unknown transformation pathways;
wherein step A comprises: collecting structural information of known antibiotics, establishing an information list of parent antibiotics, and establishing a suspect list of transformation products; and step B comprises: establishing a molecular network, extracting all features of a sub-network where a parent is located, and obtaining the transformation products from the unknown transformation pathways;
step 3: obtaining a list of candidate transformation products based on the transformation products from the known and unknown transformation pathways obtained in step 2, and annotating structures of the transformation products;
step 4: extracting feature fragments based on structure annotations of the transformation products obtained in step 3, and searching for spectra with the feature fragments, and supplementing the transformation products from the unknown transformation pathways; and
step 5: obtaining a final list of identified products after annotating the structures of transformation products.

2. The method for identifying transformation products of antibiotics from known and potential unknown transformation pathways according to claim 1, wherein:
step A comprises:
step A.1: collecting the structural information of the known antibiotics and establishing the information list of parent antibiotics;
step A.2: for a parent antibiotic obtained in step A.1, obtaining the suspect list of all transformation products; and
step A.3: merging the predicted transformation products obtained in step A.2.

3. The method for identifying transformation products of antibiotics from known and potential unknown transformation pathways according to claim 2, wherein:
in step A.2, the suspect list of the transformation products established according to the known transformation pathways comprises known transformation products and predicted transformation products from the known transformation pathways.

4. The method for identifying transformation products of antibiotics from known and potential unknown transformation pathways according to claim 2, wherein:
step A.3 comprises: removing a transformation product having a similarity of less than a set value and having the same molecular descriptor with a parent structure for merging the prediction results of the transformation products obtained in step A.2.

5. The method for identifying transformation products of antibiotics from known and potential unknown transformation pathways according to claim 1, wherein:
step B specifically comprises:
step B.1: preprocessing the mass spectrometry data obtained in step 1 to obtain a feature quantification table and a mass spectrometry (MS)/MS spectral summary;
step B.2: establishing the molecular network with the feature quantification table and an MS/MS spectral summary obtained in step B.1; and
step B.3: extracting mass spectrum peak information of transformation products of the parent antibiotic from the unknown transformation pathways using the molecular network obtained in step B.2.

6. The method for identifying transformation products of antibiotics from known and potential unknown transformation pathways according to claim 5, wherein:
step B.2 comprises: exporting the feature quantification table and the MS/MS spectral summary obtained in step B.1 to Global Natural Products Social Molecular Networking platform, creating the molecular network using a feature-based molecular networking workflow, and setting a minimum matched fragment ion and a cosine score.

7. The method for identifying transformation products of antibiotics from known and potential unknown transformation pathways according to claim 6, wherein:
step B.3 comprises: in a graph of the molecular network established in step B.2, locating an area where the parent antibiotic and the known transformation products are present based on a mass-to-charge ratio of a precursor ion shown on a node in the molecular network, thereby extracting all the features of the sub-network, and obtaining mass spectra of the transformation products of the parent antibiotic from the unknown transformation pathways.

8. The method for identifying transformation products of antibiotics from known and potential unknown transformation pathways according to claim 1, wherein:
step 3 comprises: annotating the structure of the transformation product from the unknown transformation pathways and assigning corresponding confidence levels 1 to 5 to the structures of all the transformation products.

9. The method for identifying transformation products of antibiotics from known and potential unknown transformation pathways according to claim 1, wherein:
step 4 comprises: using fragment ions frequently occurring in the parent antibiotic and transformation products screened from the samples as the feature fragments, searching the peak list with the feature fragments, and supplementing the transformation product from the unknown transformation pathways.

10. The method for identifying transformation products of antibiotics from known and potential unknown transformation pathways according to claim 8, wherein:
step 5 comprises: selecting the transformation products at the confidence levels 1 to 3 to obtain the final list of identified products.

11. The method for identifying transformation products of antibiotics from known and potential unknown transformation pathways according to claim 3, wherein:
step A.3 comprises: removing a transformation product having a similarity of less than a set value and having the same molecular descriptor with a parent structure for merging the prediction results of the transformation products obtained in step A.2.

12. The method for identifying transformation products of antibiotics from known and potential unknown transformation pathways according to claim 2, wherein:
step B specifically comprises:
step B.1: preprocessing the mass spectrometry data obtained in step 1 to obtain a feature quantification table and a mass spectrometry (MS)/MS spectral summary;
step B.2: establishing the molecular network with the feature quantification table and an MS/MS spectral summary obtained in step B.1; and
step B.3: extracting mass spectrum peak information of transformation products of the parent antibiotic from the unknown transformation pathways using the molecular network obtained in step B.2.

13. The method for identifying transformation products of antibiotics from known and potential unknown transformation pathways according to claim 3, wherein:
step B specifically comprises:
step B.1: preprocessing the mass spectrometry data obtained in step 1 to obtain a feature quantification table and a mass spectrometry (MS)/MS spectral summary;
step B.2: establishing the molecular network with the feature quantification table and an MS/MS spectral summary obtained in step B.1; and
step B.3: extracting mass spectrum peak information of transformation products of the parent antibiotic from the unknown transformation pathways using the molecular network obtained in step B.2.

14. The method for identifying transformation products of antibiotics from known and potential unknown transformation pathways according to claim 12, wherein:
step B.2 comprises: exporting the feature quantification table and the MS/MS spectral summary obtained in step B.1 to Global Natural Products Social Molecular Networking platform, creating the molecular network using a feature-based molecular networking workflow, and setting a minimum matched fragment ion and a cosine score.

15. The method for identifying transformation products of antibiotics from known and potential unknown transformation pathways according to claim 13, wherein:
step B.2 comprises: exporting the feature quantification table and the MS/MS spectral summary obtained in step B.1 to Global Natural Products Social Molecular Networking platform, creating the molecular network using a feature-based molecular networking workflow, and setting a minimum matched fragment ion and a cosine score.

16. The method for identifying transformation products of antibiotics from known and potential unknown transformation pathways according to claim 14, wherein:
step B.3 comprises: in a graph of the molecular network established in step B.2, locating an area where the parent antibiotic and the known transformation products are present based on a mass-to-charge ratio of a precursor ion shown on a node in the molecular network, thereby extracting all the features of the sub-network, and obtaining mass spectra of the transformation products of the parent antibiotic from the unknown transformation pathways.

17. The method for identifying transformation products of antibiotics from known and potential unknown transformation pathways according to claim 2, wherein:
step 3 comprises: annotating the structure of the transformation product from the unknown transformation pathways and assigning corresponding confidence levels 1 to 5 to the structures of all the transformation products.

18. The method for identifying transformation products of antibiotics from known and potential unknown transformation pathways according to claim 3, wherein:
step 3 comprises: annotating the structure of the transformation product from the unknown transformation pathways and assigning corresponding confidence levels 1 to 5 to the structures of all the transformation products.

19. The method for identifying transformation products of antibiotics from known and potential unknown transformation pathways according to claim 2, wherein:
step 4 comprises: using fragment ions frequently occurring in the parent antibiotic and transformation products screened from the samples as the feature fragments, searching the peak list with the feature fragments, and supplementing the transformation product from the unknown transformation pathways.

20. The method for identifying transformation products of antibiotics from known and potential unknown transformation pathways according to claim 3, wherein:
step 4 comprises: using fragment ions frequently occurring in the parent antibiotic and transformation products screened from the samples as the feature fragments, searching the peak list with the feature fragments, and supplementing the transformation product from the unknown transformation pathways.
